(19) United States
(12) Patent Application Publication
(10) Pub. No.: US 2025/0366834 A1
(43) Pub. Date: Dec. 4, 2025

(54) FASTENING ARM FOR URINE ANALYSIS DEVICE

(71) Applicant: Withings, ISSY LES MOULINEAUX (FR)

(72) Inventor: Arthur PONS, ISSY LES MOULINEAUX (FR)

(21) Appl. No.: 19/219,442

(22) Filed: May 27, 2025

(30) Foreign Application Priority Data
May 30, 2024 (FR) FR2405614

(51) Int. Cl. A61B 10/00; E03D 9/00; G01N 33/493

(52) U.S. Cl. CPC A61B 10/007; E03D 9/00; G01N 33/493

(57) ABSTRACT

A urine analysis kit includes a urine analysis device configured for placement entirely within a toilet bowl and including a housing and an attachment member disposed on a rear face of the housing; a fastening arm configured to position the urine analysis device entirely within the toilet bowl, the fastening arm including a proximal attachment portion configured to cooperate with the attachment element of the urine analysis device; the proximal attachment portion forming a receiving space for the housing, wherein the receiving space extends from the rear face to beyond an edge of the rear face.

### CROSS-REFERENCE TO RELATED APPLICATIONS

This application claims priority to French Patent Application No. FR2405614, filed May 30, 2024, the entire content of which is incorporated herein by reference in its entirety.

### FIELD

The present invention relates to a urine analysis assembly comprising a urine analysis device. The urine analysis device comprises a housing configured to be placed entirely within a toilet bowl. The assembly further comprises a fastening arm for securing the urine analysis device to the toilet bowl.

### BACKGROUND

Many biological parameters can be identified in an individual's urine. For example, health problems such as urinary tract infection, diabetes or kidney failure can be detected from a urine sample. The urine sample can also reflect the quality of a diet, identify a period of fertility or pregnancy, and detect drug or tobacco use. It therefore makes sense to periodically monitor various biological parameters.

It is known to offer toilet devices with a urine analysis function. These devices are capable of taking urine samples from the toilet and analyzing them to determine the level of a biological parameter.

Such a urine analysis device is described in document WO2021/175909. This document presents a device for analyzing urine. The device fits into a toilet and collects a urine sample before performing an optical analysis on a test support. The device comprises a station and a cartridge, also known as a rotating holder, which can be removed and replaced from the station. The cartridge contains urine strips, i.e. strips coated or impregnated with a reagent that reacts with urine.

A number of solutions have been proposed for placing such a urine analysis device in a toilet. Document WO2021/175909, for example, proposes a suction cup on the rear face of the device's housing to cooperate with the toilet wall (paragraph 106), or a hook mounted on a rim of the toilet bowl (paragraph 110). Document WO2023/036805 suggests magnets or an adhesive surface on the rear of the housing to cooperate with the inner wall of the toilet (paragraph 93). At the Consumer Electronics Show (CES) in Las Vegas in January 2023, a urine analysis device with a hook was presented. The urine analysis device comprises a housing with a rear face, facing a toilet bowl wall. On the rear face, a pin is positioned; this pin cooperates with an orifice of an arm, which then hooks onto the rim of the toilet bowl. Between the hole and the hook, the arm comprises two straight portions, with a slight change in direction between the two portions.

Document GB2546847 describes a urine test with a colorimetric detection portion (i.e. a material coated with a chemical reagent to detect compounds in urine) temporarily attached to the toilet bowl by a hook. The user places the urine test in the toilet just before urinating, urinates directly onto the detection portion, then removes it from the toilet to visually compare the colorimetric detection portion with a reference

However, these various solutions however encounter a problem due to the different shapes of toilet bowls, which can differ significantly from country to country. For example, some toilet bowls have a shoulder close to the rim of the toilet bowl, which can touch the hook arm and hinder the proper installation of the urine analysis device. However, when the urine analysis device is to remain in the toilet for several weeks at a time, it is imperative that the attachment is optimized.

### SUMMARY

The present description therefore proposes a urine analysis kit for easy and compatible installation in various toilet bowl shapes.

To this end, the present description relates to a urine analysis kit comprising:
a urine analysis device configured to be placed entirely within a toilet bowl and comprising:
   a housing, the housing comprising:
      a front face designed to receive a stream of urine from a user urinating in the toilet bowl, and
      a rear face opposite the front face,
   an attachment element on the rear face of the housing,
a fastening arm configured to position the urine analysis device entirely within the toilet bowl, the fastening arm comprising a proximal attachment portion configured to cooperate with the attachment element of the urine analysis device; wherein the proximal attachment portion forms a receiving space for the housing, wherein the receiving space extends from the rear face to beyond an edge of the rear face.

In fact, such a urine analysis assembly provides a urine analysis device offering a front face that can be blank to receive a jet of urine and an attachment element arranged on the rear face, the attachment element thus being hidden and protected. The fastening arm enables the urine analysis device to be fixed in toilet bowls by cooperating with the attachment element. The shape of the fastening arm, which forms a receiving space for the housing from the attachment element to the top, allows the arm to conform to the shape of the housing and thus enables a stable and solid cooperation. In addition, this shape allows the portion of the fastening arm extending between the housing and the rim of the bowl to be offset away from the bowl, thus avoiding contact which could lead to incorrect positioning of the urine analysis device. In this way, the Urine analysis Set adapts easily to different types of toilet bowl shapes. So, unlike a straight arm which may come into contact with the bowl wall and force the urine analysis device into an unsuitable position, the fastening arm as described here enables proper positioning of the urine analysis device. In particular, the fastening arm as described herein provides a spatially offset and mechanically stable configuration that avoids interference with irregular toilet bowl geometries (e.g., shoulders, contours, or projections), thereby improving compatibility, durability of attachment, and accuracy of urine collection due to optimal sensor alignment. The receiving space extends from the rear face to beyond the upper edge of the housing, thereby partially enveloping the housing's upper contour. This configuration not only stabilizes the housing within the toilet bowl but also mechanically restricts rotational and translational displacements under lateral forces or flushing turbulence.

In an embodiment, the rear face is designed to face a wall of the bowl.

In an embodiment, the fastening arm is removable.

In an embodiment, the fastening arm is flexible. As used herein, the term “flexible” refers to the ability of the fastening arm to elastically deform in at least one direction (for example in the median plane XZ) in response to external forces such as manual installation pressure or the gravitational weight of the urine analysis device. Such flexibility allows the arm to conform to variable toilet geometries without permanent deformation, enabling a resilient fit that ensures proper positioning and reliable contact of the housing with the inner wall of the toilet. In some embodiments, flexibility may result from the use of polymeric materials (e.g., nylon, polyethylene) or thin metal structures with an elastic modulus suitable for temporary deflection.

In an embodiment, the fastening arm has a flattened shape in a section transverse to the median plane. As used herein, the term “flattened” refers to a cross-sectional geometry in which one dimension (e.g., height along the Z axis) is substantially greater than the other (e.g., thickness along the Y axis), such that the section has an oblong, oval, or generally plate-like appearance. This geometry imparts directional flexibility to the fastening arm-allowing it to bend more easily within the median plane XZ—while maintaining lateral stiffness in the transverse direction Y. This controlled anisotropic flexibility improves both adaptability to curved toilet surfaces and mechanical stability.

In an embodiment, the fastening arm has a width of between 0.5 cm and 20 cm.

In an embodiment, the proximal attachment portion extends in particular between a first end and a second end, wherein the first end is flush with the attachment element on the rear face and the second end is beyond the edge of the rear face.

In an embodiment, the first end extends to the front face of the housing.

In an embodiment, the second end comprises a stop configured to block movement of the urine analysis device otherwise permitted by the attachment element.

In an embodiment, the receiving space is configured to at least partially receive the upper part of the rear face of the housing, up to the top of the housing.

In an embodiment, the receiving space is complementary in shape to the housing, in particular with a functional clearance.

In an embodiment, the attachment element is arranged in the upper part of the rear face, for example between 15% and 25% from the edge of the rear face.

In an embodiment, the attachment element is chosen from: a lug, a magnet, an adhesive part.

In an embodiment, when the attachment element is a stud, the first end portion comprises a cavity (beneficially through-going) designed to receive the attachment element.

In an embodiment, the lug extends from the housing along a body and then a head that is wider than the body.

In an embodiment, the cavity has a first section of maximum transverse width greater than the width of the head and a second section of maximum transverse width less than the width of the head.

In an embodiment, the fastening arm further comprises a distal attachment portion configured to cooperate with the toilet bowl.

In an embodiment, the distal attachment portion comprises a hook/magnet/adhesive part.

In an embodiment, the fastening arm further comprises an intermediate portion arranged between the proximal attachment portion and the distal attachment portion.

In an embodiment, the proximal attachment portion is connected to the intermediate portion at the second end.

In an embodiment, the fastening arm has a change of direction between the intermediate portion and the proximal attachment portion.

In an embodiment, the intermediate portion and the proximal attachment portion form an angle (a) of less than 90°, in particular less than 60°, with each other at the point of contact.

In an embodiment, the stop is formed by the change in direction between the proximal attachment portion and the intermediate portion.

In an embodiment, the intermediate portion and the proximal attachment portion form an inflection zone between them.

In an embodiment, the intermediate portion and the proximal attachment portion form a ridge between them.

In an embodiment, the intermediate portion extends as an extension of the housing in the median plane of the housing.

In an embodiment, the intermediate portion extends from the front face in the mid-plane of the housing.

In an embodiment, the upper portion comprises a rear surface facing the bowl and a front surface opposite the rear surface, the front surface of the intermediate portion extending in line with the front face of the housing.

In an embodiment, the intermediate portion has a rounded shape.

In an embodiment, the intermediate portion has a convex shape.

In an embodiment, the convex shape tends to move the device away from the bowl.

In an embodiment, the urine analysis device comprises a collection port, the collection port being configured to collect liquid from outside the housing to inside the housing.

In an embodiment, the collection port is located on the rear face.

In an embodiment, the rear face comprises at least one spacer projecting from the rear face and configured to contact the toilet bowl.

The present description also relates to a fastening arm configured to position a urine analysis device entirely within a toilet bowl, the analysis device comprising a housing comprising: a front face for receiving a stream of urine from a user urinating into the toilet bowl, and a rear face opposite the front face; and an attachment member disposed on the rear face of the housing, the fastening arm comprising a proximal attachment portion configured to cooperate with the attachment member of the urine analysis device; wherein the proximal attachment portion forms a receiving space for the housing, wherein the receiving space extends from the rear face to beyond an edge of the rear face.

### BRIEF DESCRIPTION OF THE DRAWINGS

Further features, details and benefits will become apparent from the detailed description below, and from an analysis of the appended drawings, in which:

FIG. 1 shows a simplified schematic representation of a urine analysis device installed in a toilet bowl,

FIG. 2 shows an exploded view of the urine analysis device, in which the station and cartridge are visible,

FIG. 3 shows a detailed view of a cartridge In an embodiment,

FIG. 4 shows a cross-sectional view of a cartridge and station according to one embodiment, at the location of an optical analyzer of the station,

FIG. 5 shows a side view of an analyzer assembly according to an embodiment of the description with a fastening arm in a resting configuration,

FIG. 6 shows a side view of an analysis assembly according to an embodiment of the description with a fastening arm in a deployed configuration, [FIG. 7]: FIG. 7 shows a side view of an analysis assembly according to an embodiment of the description with a fastening arm in a deployed configuration,

FIG. 7 shows a perspective view (a) and a side view (b) of a fastening arm of the assembly shown in FIGS. 5 and 6,

FIG. 8 shows a partial side view (a) of a urine analysis device and a front view (b) of the fastening arm of the assembly shown in FIGS. 5 and 6,

FIG. 9 shows a side view of an analysis assembly according to another embodiment of the description.

### DETAILED DESCRIPTION

The present description presents various examples of a urine analysis device comprising a station and a cartridge as disclosed in documents WO2021/175909 and WO2021/175944, hereinafter referred to as WO′909 and WO′944. Variations of the stations are disclosed in documents WO2023036805, WO2023036806, WO2023036808, WO2023036809, hereinafter referred to as WO′80X.

The following paragraphs explain the general principle of a urine analysis device, but all the details of documents WO′909 and WO′933 (as well as all the above-mentioned PCT documents) are applicable.

### Orientation

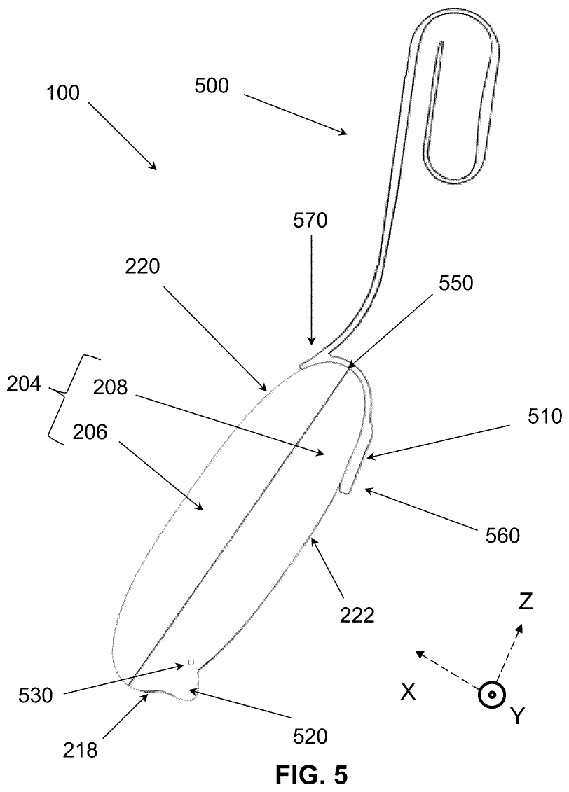

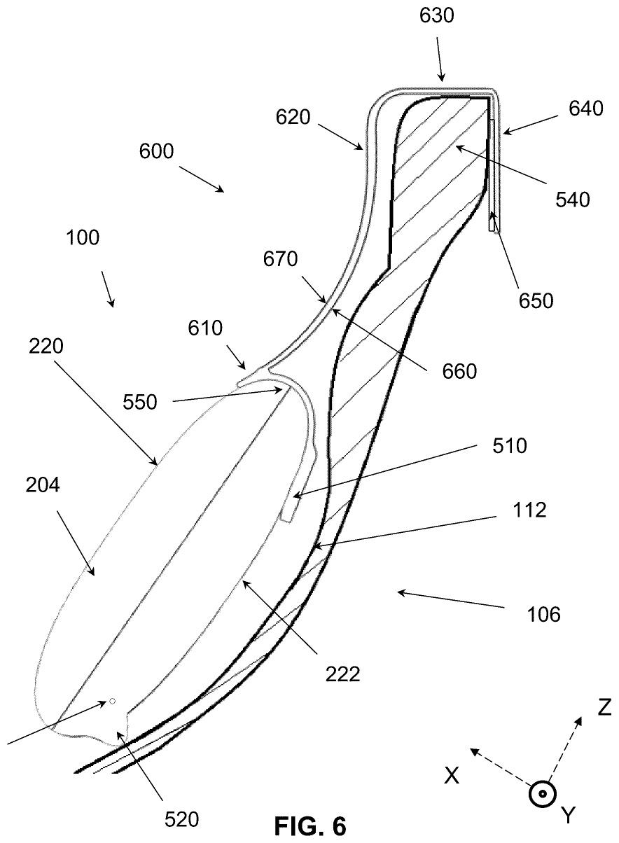

In the following, the notions of “top” and “bottom”, “upper” and “lower”, etc. are defined in relation to a Z direction, as defined in FIGS. 5 and 6. The top along the Z direction is defined in a normal use position of the urine analysis device fixed in the toilet bowl.

### Overall Shape of Housing

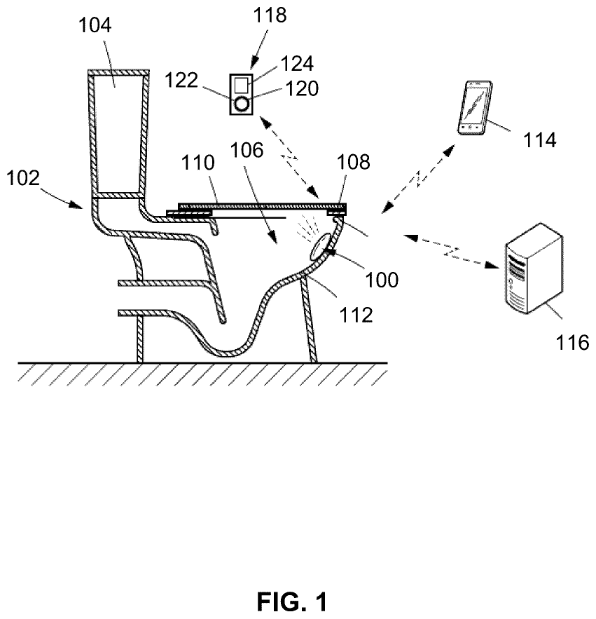

FIG. 1 schematically illustrates an analysis device 100 (referred to as “device 100”) for urine analysis installed in a toilet 102. Toilets 102 typically comprise a water tank 104, a bowl 106, a seat 108 and a seat cover 110. The analysis device 100 is configured to be placed entirely within the toilet bowl. By “in the bowl” is meant “placed in the interior volume defined by the bowl”. The analysis device 100 is removably arranged in the toilet 102. For example, the analysis device 100 can be easily removed from the toilet to replace a cartridge, and then replaced in the toilet 102. The analysis device 100 is placed on an inner wall 112 of the toilet bowl 106. The analysis device 100 is positioned so that it is generally under a user's urine stream, so that when a user urinates (generally in a seated position), the urine comes into contact with the 100 analysis device. The analysis device 100 can communicate remotely with a remote entity, such as the smartphone 114 or a server 116.

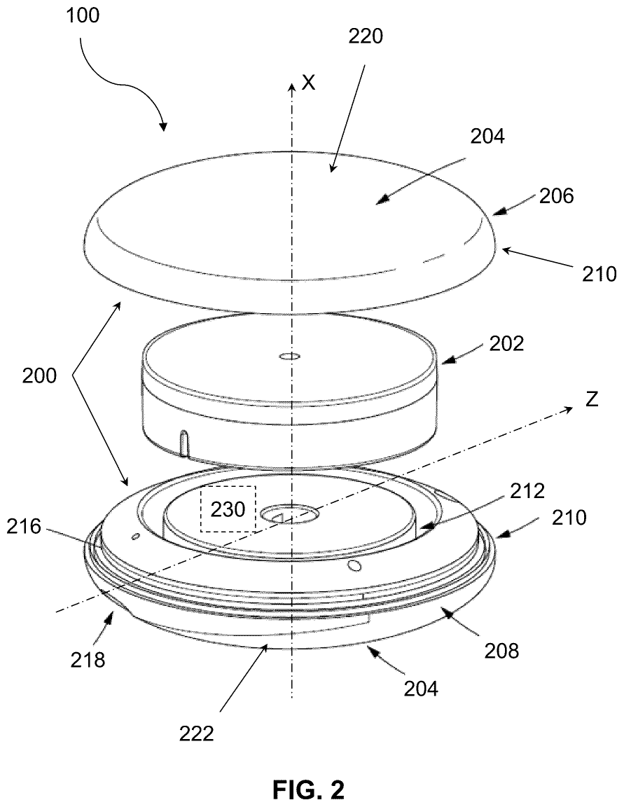

As illustrated in greater detail in FIG. 2, the analysis device 100 may comprise a station 200 and a cartridge 202, removably mounted on the station 200. The station 200 may comprise a housing 204 which may comprise two shells, in particular a front shell 206 and a rear shell 208. The front shell 206 and the rear shell 208 may cooperate with each other via a fastening mechanism 216, in a plane normal to the X axis. The front shell 206 and rear shell 208 can be reversibly assembled, for example by screwing or clipping. Alternatively, the front shell 206 and rear shell 208 can be permanently joined, for example by gluing, clipping, magnetizing or ultrasonic welding. It will be appreciated that other fastening methods can also be used to join the two shells together.

In particular, as shown in FIG. 2, the front shell 206 and the rear shell 208 are screwed together. Then, an internal part of the front shell 206 comprises a thread. The thread on the front shell 206 is designed to cooperate with a complementary thread on the rear shell 208. This allows the housing 204 to be easily disassembled to gain access to the test assembly inside the housing.

A seal may be present between the front shell 206 and the rear shell 208. In this way, the housing 204 is watertight. Only collection and drainage ports connect the outside and inside of housing 204, as described in more detail below.

As can be seen from the figures, the housing 204 may have the overall external shape of a circular roller. In other words, the housing 204 has a spheroidal shape. The X axis is the center line of the housing. Beneficially, the front shell 206 can be substantially rotationally symmetrical, giving the device an aerodynamic appearance once installed. The housing 204 serves as a urine collector.

The housing 204 comprises a front face 220 for receiving a stream of urine directly from a user urinating on the toilet, and a rear face 222 opposite the front face 220. As illustrated in FIG. 2, the front face 220 can be arranged on the front shell 206 and the rear face 222 can be arranged on the rear shell 208. The front face 220 faces the inside of the bowl 106. The front face 220 is therefore intended to receive urine when the user urinates while sitting on the toilet 102. As shown in FIGS. 5 and 6, the rear face 222 faces the inner wall 112 of the bowl 106. For the rest of the description, by an object facing the bowl wall is meant an object facing the bowl wall closest to the object in question, and not the facing bowl wall on the other side of the inner bowl volume.

The front face 220 and rear face 222 may each have a curved edge 210. The respective curved edges 210 of the front and rear faces meet at an equatorial junction zone. In this way, the outer surface of housing 204, consisting of front face 220 and rear face 222, is defined by curved lines and forms a generally convex object.

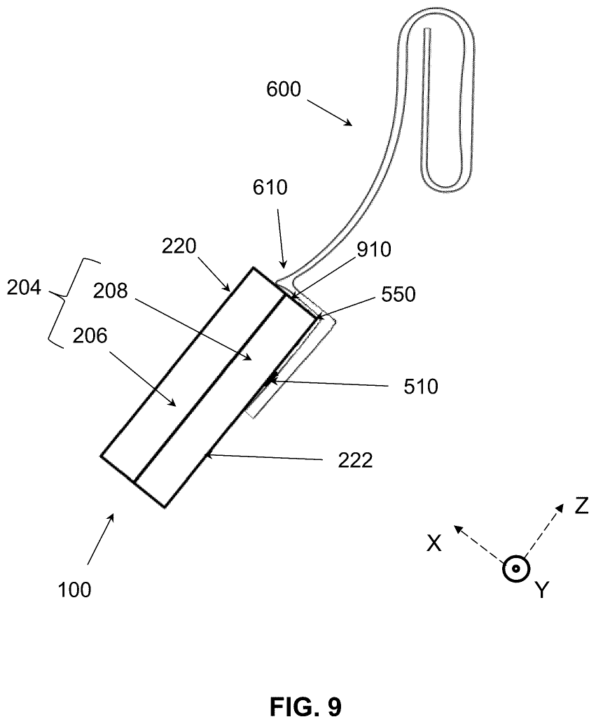

With reference to FIGS. 5 and 6, the rear face 222 has an edge 550. Edge 550 is where the rear face ends and either a transition face or the front face begins. The edge 550 typically passes close to a highest point of the housing 204 along the Z axis in a normal use position of the housing 204 when placed in the toilet bowl 106. In the two-shell pebble design, edge 550 is at the junction of front face 220 and rear face 222. In a parallelepipedal version of housing 204, as shown in FIG. 9, edge 550 is located at the junction between the rear face 222 and an upper face 910, which connects the front face 220 and the rear face 222

The outer surface of the front panel 220 may be smooth. In other words, the front face 220 is free of ridges or grooves. In this way, the flow of urine coming into contact with the front face 220 catches and spreads over the front face 220. The front face 220 can be substantially rotationally symmetrical about the X axis.

The outer surface of housing 204 can also be white or light-colored. The color of the outer surface can be similar to that of the toilet, which enhances the discreetness of the device.

Housing 204 can have a diameter, measured in the direction orthogonal to axis X, of between 50 mm and 150 mm. The housing 204 can have a thickness, measured in the direction of the X axis, of between 15 mm and 50 mm. In this way, housing 204 is sufficiently compact to be fully housed in the toilet bowl. The urine analysis 100 device is unobtrusive. In addition, housing 204 is large enough to systematically come into contact with the urine received in the toilet bowl. The user can then urinate in the toilet without worrying about the urine analysis device, or alternatively aim summarily.

According to another aspect, In an embodiment, the housing 204 has a general form factor such that the ratio between thickness and diameter is between 0.2 and 0.5, and in an embodiment between 0.3 and 0.4. Such proportions are reminiscent of a natural pebble and give the device a soothing appearance. The spheroidal ‘pebble’ shape minimizes splash-back and offers low resistance to water flow, encouraging complete and uniform flushing.

In an embodiment, housing 204 is made of a hydrophilic material. For example, the material of housing 204 may be: ceramic, polyamide (PA), silicone or a hydrophilic polymer. The outer surface of housing 204 can also be treated with a hydrophilic surface treatment, such as AcuWet® from Aculon, a hydrophilic polymer, or Pebax® from Arkema.

### Test Set

A test assembly 230 is located inside housing 204 and configured to perform an analysis on urine collected through the collection port. Station 200 comprises an annular compartment 212, located inside housing 204, arranged around an axis of rotation X. The annular compartment 212 is configured to at least partially receive the cartridge 202 mounted rotatably around the axis of rotation X (once in position in the annular compartment 212). The cartridge 202 comprises a plurality of test supports, each of which comprises at least one urine reagent, for example a dry reagent, the plurality of test supports being arranged along a circle or arc of a circle around the axis of rotation X. In an embodiment, the test supports are test strips. The test supports can be enclosed, for example individually, in a chamber.

The annular compartment 212 typically extends over 360° and forms a groove configured to at least partially receive the cartridge 202.

Station 200 comprises a collection port 218, located for example on the rear shell 208. As will be explained in more detail below, the collection port 218 is configured to collect urine flowing over the surface of the housing 204. Station 200 also comprises a drain port 530, visible in FIG. 5, configured to drain liquid out of device 100. The rear-facing collection port and spacer arrangement prevents direct exposure to user urine streams and flush surges, reducing fouling risk and ensuring sensor longevity. This also avoids turbulent flow disruption during sample intake, improving test accuracy

The test assembly 230 may comprise a pump, an injector and an analyzer. The pump draws urine from collection port 218, then the injector injects the urine onto one or more test media in the cartridge, and the analyzer obtains certain property values (for example, physical/chemical properties, such as color) from the test media after they have come into contact with the urine. In an embodiment, the analyzer is an optical analyzer configured to analyze the optical properties of the test media. The injector and cartridge can move relative to each other so that the injector can open (e.g. pierce) the chamber, for example using a needle or needle-like device.

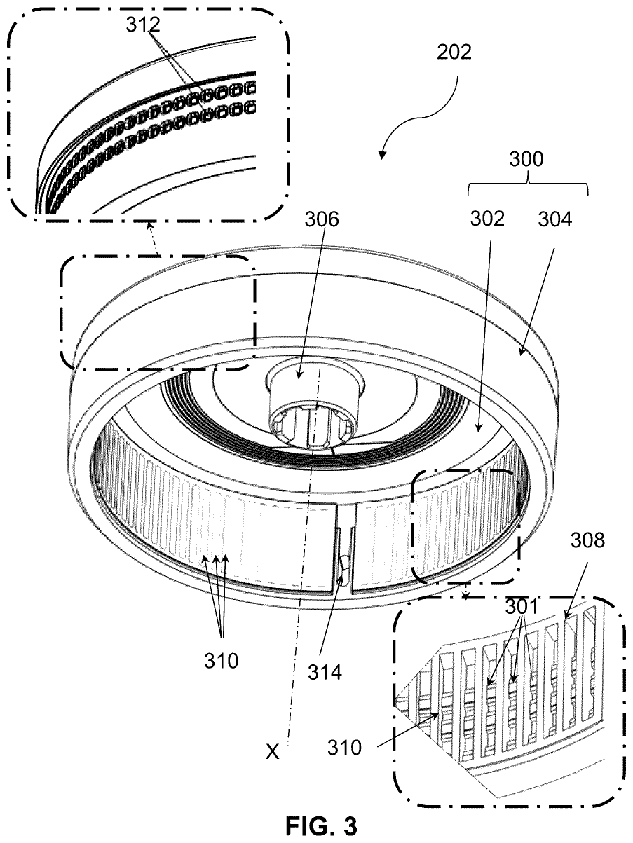

FIG. 3 shows an exploded view of cartridge 202. Cartridge 202 comprises at least one test support 301, in particular several test support 301 configured to receive urine from the injector. Each test support 301 contains a urinary reagent which reacts in a specific way on contact with urine. The cartridge 202 comprises a rotating support 300, configured to be rotated by the station 200. During normal use of the cartridge 202 and the device 100, the test supports 301 remain attached to the rotating support and do not move relative to it.

In an embodiment, the rotating support 300 has a straight circular cylinder shape of at least 80% of a hollow cylinder shape extending annularly around an axis which is, when the cartridge 202 is mounted in the station 200, the axis of rotation X. Each test support 301 may be a test strip. The rotating support 300 may comprise an annular portion 302 and a cylindrical portion 304, which extends from a radially outer end of the annular portion 302. The cylindrical portion 304, when in use, is housed inside the annular compartment 212. The test supports 301 are positioned along the cylindrical portion 304, so as to be able to scroll selectively and/or successively past the injector and analyzer. For example, the test supports 301 are part of a holder 308, which comprises several chambers 310, separated from each other along a perimeter around the X axis. At least one test strip is received in a chamber 310.

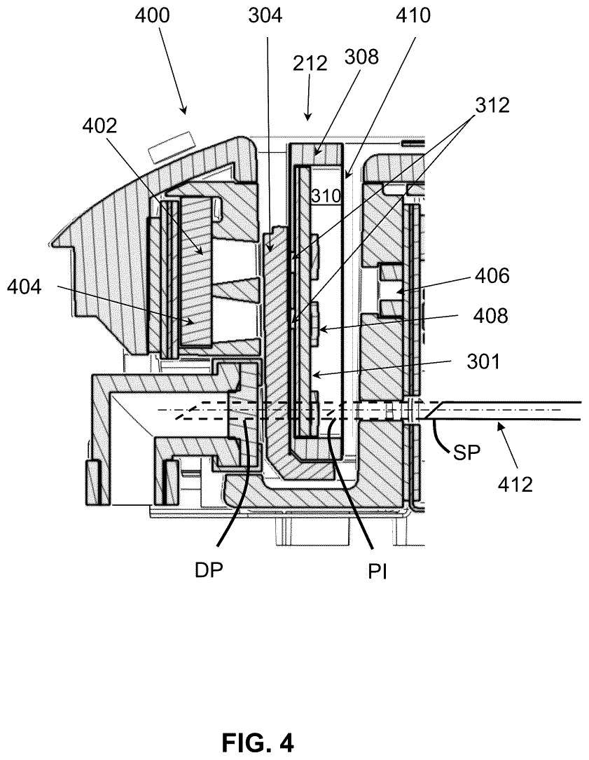

The chambers 310 are arranged side by side in the shape of a right circular cylinder of at least 80% of the circle. To allow light to pass through, the holder 308 comprises at least one opening 312 per chamber 310 (shown in the upper left zoom where the rotating support is represented as transparent). The chambers 310 are all equidistant from the axis of rotation X, so that the injector can selectively inject urine once the desired chamber is positioned at the desired location facing the injector. The injector can move towards chamber 310 and pierce a cover closing chamber 310 (visible in FIG. 4). A drain hole 314 is provided in the rotating support 300 to allow urine to drain from the injector to the outside of the device 100, via the drain port 530 located on the housing 204.

The annular portion 302 of the rotary support 300 remains outside the annular compartment 212 to reinforce the cylindrical part and/or drive the cartridge 202 in rotation. To this end, the annular portion 302 may comprise a mechanical coupling 306, which cooperates with a shaft of the station 200.

The dimensions of cartridge 202 are disclosed in documents WO′909, WO′933 and WO′80X. The maximum dimension of the device 100 transverse to the axis of rotation X is less than 15 cm, or even less than 10 cm. The maximum dimension of the device along the axis of rotation X is less than 5 cm.

FIG. 4 shows in more detail the interaction between cartridge 202 and station 200 when or after injector activation. The analyzer 400 comprises at least one light source 402, 404 (for example, two light sources) and at least one optical sensor 406. Light travels from the light source 402, 404 to the optical sensor 406, passing through the cartridge 202 and in particular the cylindrical portion 304, the opening 312 of the holder 308, the test support 301 and thus the reagent 408.

In an embodiment, the analyzer 400 is configured to measure the absorbance of a portion of the test support 301.

Absorbance is detected by the light source (e.g. an LED), which can pass light through the strip, and the optical sensor, which receives the spectrum at around ten wavelengths.

In an embodiment, the light sensor is a camera capable of detecting a change in color, in particular a change in color intensity, of a portion of the test support 301.

The camera can detect color in RGB values, for example.

The injector comprises an injection end 412 (e.g. a needle), which can be moved between several positions, represented by dashed lines in FIG. 4. In a stand-by position SP, the injection end 412 is outside the cartridge 202 (in an innermost position), so that the cartridge 202 can rotate freely in the annular compartment 212; in an injection position IP, injection end 412 has pierced cover 410 to gain access to the inside of chamber 310 and is able to inject some urine onto test support 301; in the discharge position DP, injection end 412 is able to discharge urine through drain hole 314 of rotating support 300.

In the SP position, the injector is located radially inside the annular chamber. This maximizes the radius of the annular chamber while minimizing the size of station 200.

In an embodiment of the test assembly 230 disclosed above, the skilled person will understand that each test set configured to analyze the urine sample collected through collection port 218 may be disposed within housing 204.

### Collection Port

Collection port 218 is configured to receive urine flowing by gravity over the outer surface of housing 204. Urine is collected directly on the front face 220 and rear face 222 of housing 204.

The collection port 218 is an opening configured to collect the liquid, so that the liquid can enter the urine analysis device. The collection port 218 is generally circular, with a diameter of, in an embodiment, between 0.3 mm and 2 mm. The diameter of the collection orifice can be chosen to maximize the volume of urine collected on the outer surface of housing 204.

As can be seen from the figures, the collection port 218 is located on the rear face 222. In this way, the collection port 218 faces the inner wall 112 of the toilet when the urine analysis device 100 is positioned in the toilet. This position allows the collection port 218 to be hidden from the user's view by the front face 220 of the housing. The front face 220 visible to the user resembles a simple, uniform roller, as already mentioned, with no singular points or holes. It should also be noted that this position prevents the introduction of contaminants or elements that could obstruct the 230 test assembly.

The collection port 218 is located on a lower part of the rear face 222. By “on a lower part of the rear face”, we mean “on the last quarter of the face along the Z direction from the lower end of the housing 204”. The lower end faces the bottom of bowl 106 when housing 204 is positioned in the bowl. The lower end is opposite the edge 550. This position corresponds to normal use. This position allows urine to be collected by gravity over most of the outer surface of housing 204.

In particular, the distance separating the collection port 218 from a lower edge of the housing 204 is less than 40 mm, such as less than 20 mm. As illustrated, according to a particular embodiment, the collection port 218 is arranged a few millimeters above the lower edge of the housing 204. Alternatively, the collection port 218 may be located on the bottom edge (the bottom edge being defined when the device 100 is placed for use in a toilet).

The collection port 218 can be covered by a mesh filter. The mesh filter is, for example, oblong in shape and covers the collection port 218. The average mesh size of the filter is, for example, 20 microns. The mesh filter prevents the introduction of contaminants or elements likely to clog the test set 230 and filters the urine received in the collection port 218. The filter mesh may be made of metal.

### Spreader

Referring in particular to FIGS. 5 and 6, in an embodiment the device 100 may comprise at least one spacer 520 projecting from the rear face 222 and configured to be in contact with the bowl 106, in particular in contact with the inner wall 112 of the bowl 106. The spacer 520 is located on the rear face 222. In an embodiment, the spacer 520 is arranged on the rear face 222 so that the collection port 218 is at a certain distance from the bowl 106 during normal use of the device. This prevents the inner wall 112 of the bowl 106 from obstructing the inlet to the collection port 218 and/or forming a bottleneck for the flow of a mixture of urine, water and impurities from the bowl (which could otherwise be collected by the collection port).

Thanks to the at least one spacer 520, urine flows over the housing 204 (in particular on the rear face 222) towards the collection port 218 without being obstructed by the bowl 106 as could happen with a device as described in documents WO′909 and WO′944.

In an embodiment, the spacers 520 are located on the lower part of the urine analysis device 100, and more particularly on the lower part of the rear face 222, next to the collection port 218.

The spacer 520 typically comprises an edge. In use, part of the edge is in contact with the toilet bowl.

In particular, the device 100 may comprise two spacers 520, beneficially arranged on either side of the collection port 218, along the transverse direction Y. The two spacers 520 may be arranged on either side of the collection port 218, along the collection port 218. The two spacers 520 may be symmetrical, for example along a vertical plane XZ, in a normal use position of the device 100 or, for example, around the collection port 218.

The spacer 520 is described in detail in patent application EP23192265.

### FIXING

The housing 204 is designed to be placed on the inner wall 112 of the bowl 106. The housing 204 is positioned and held in position in the toilet bowl by a fastening arm 600. The fastening arm 600 is configured to secure the urine analysis device 100 in the toilet bowl 106. The fastening arm 600 comprises several sections which have different functions and will be described in detail later. However, the fastening arm 600 is typically made from a single material, such as plastic, by molding.

The analysis device 100 comprises an attachment element 510 arranged on the rear face 222 of the housing. The attachment element 510 is configured to cooperate with the fastening arm 600. With an attachment element 510 on the rear face 222, the front face 220 can be left blank and thus prevent the urine jet from being accidentally reflected towards the toilet bowl opening. Indeed, the distance between the urethra and the urine analysis device, as well as the pressure of the urine jet, mean that any asperity on the front face 220 receiving the jet can cause splashing.

The attachment element 510 is arranged, for example, at a median plane XZ of the housing 204. In other words, the attachment element 510 is arranged in a vertical plane of symmetry of the housing 204.

As can be seen from the figures, the attachment element 510 can be arranged on the upper part of the housing 204. By “on the upper part of the housing”, we mean “on the first quarter along the Z direction from the top of the housing 204”. In particular, the attachment element 510 is arranged between 15% and 25% of the rear face 222 starting from the edge 550 of the housing 204. For example, the attachment element 510 is located within a vertical band that begins approximately 15% and ends approximately 25% of the total height of the rear face 222, as measured from the upper edge 550 in the direction toward the lower end of the housing. This positional range ensures consistent placement of the fastening arm and facilitates mechanical engagement without interfering with fluid collection functions or toilet bowl geometry.

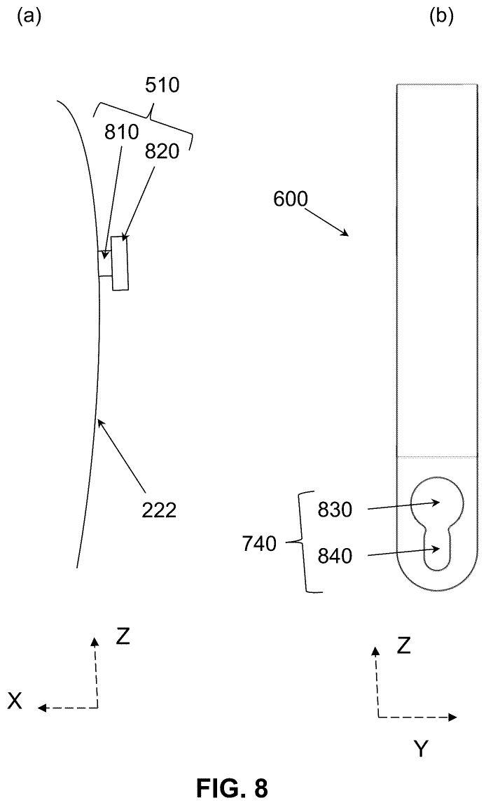

As shown in FIG. 8, the attachment element 510 may be a pin. The lug protrudes from the rear face 222 of the housing 204. In an embodiment, the lug extends from the rear face 222 through a body 810 and then through a head 820 of greater transverse extent than the body 810.

In a variant not shown, the attachment element 510 is a magnet suitable for cooperating with another magnet placed on the fastening arm 600.

In a variant not shown, the attachment element 510 comprises an adhesive part suitable for cooperating with the fastening arm 600.

The proximal attachment feature, whether implemented via a lug, magnet, or adhesive, allows secure yet reversible installation. This facilitates maintenance, particularly cartridge replacement or cleaning, without requiring removal of the entire fastening assembly.

### Mounting Arms

In an embodiment, the fastening arm 600 is removable from the urine analysis device. In other words, the user can separate the fastening arm 600 from the attachment element 510. In this way, the fastening arm 600 can be easily replaced, and another form of fastening arm can be used, for example.

The fastening arm 600 can be flexible. In other words, fastening arm 600 can be deformed by the user or by the gravity of the device 100 placed in the toilet. This makes it easier to adapt the mounting arm to different toilet shapes, and ensures that the housing 204 touches the inner wall 112 of the toilet. In particular, fastening arm 600 is made of a plastic material, e.g. nylon, or a metal material, e.g. steel.

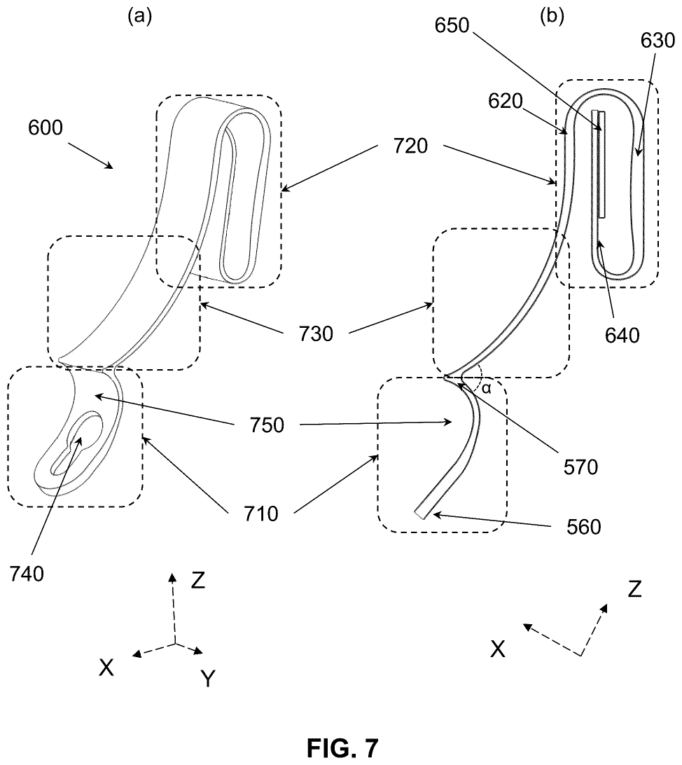

As shown in FIG. 7, the fastening arm 600 can have a flattened shape in a section transverse to the median plane XZ. This allows flexibility of the fastening arm 600 in the median plane XZ, while limiting flexibility in the transverse direction Y.

Fastening arm 600 can extend to a height along the Z axis of between 5 cm and 15 cm. Fastening arm 600 may have a width along the Y axis of between 1 cm and 2 cm. Fastening arm 600 may have a thickness of between 1 mm and 2 mm.

### Proximal Attachment Portion

With reference to FIGS. 6 and 7, the fastening arm 600 comprises a proximal attachment portion 710 configured to cooperate with the attachment element 510 of the urine analysis device 100. Hereafter, the terms “proximal” and “distal” are defined according to proximity to the analysis device 100.

When the attachment element 510 is a pin, the proximal attachment portion 710 comprises a cavity 740 designed to receive the attachment element 510. Cavity 740 may be continuous.

With reference to FIG. 8, cavity 740 can have a first section 830 with a maximum transverse width greater than the width pin head 820 and a second section 840 with a maximum transverse width less than the width of pin head 820. The second section 840 also has a greater transverse width than the lug body 810. So, when the user wants to assemble the fastening arm 600 with the device 100, he/she inserts the pin into the first section 830 and then slides the pin into the second section 830. As the head 820 of the pin is wider than the second section 830, the fastening arm 600 is held fast to the device 100. Conversely, when the user wishes to disengage the fastening arm 600 from the device 100, he/she slides the pin from the second section 840 towards the first section 830 to allow the head 820 of the pin to pass through the cavity 740.

The proximal attachment portion 710 comprises a first end 560 at the attachment element 510 and a second end 570 which is beyond the edge 550 of the rear face 222 (when the fastening arm 600 is associated with the housing 204). In an embodiment, the second end 570 is at the level of the front face 220 of the housing 204. As illustrated in the figures, the proximal attachment portion 710 extends essentially between these two ends 560, 570.

### Reception Area

As shown in FIGS. 5 to 9, the proximal attachment portion 710 forms a receiving space 750 for the housing 204. When the fastening arm 600 cooperates with the housing 204, the receiving space 750 extends from the attachment element 510 to at least one edge 550 of the rear face 222 of the housing 204. The receiving space 750 thus extends from the rear face 222 to at least the edge 550. In an embodiment, receiving space 750 extends to the front face 220 of housing 204, as shown in FIG. 6. The receiving space 750 is thus delimited by the first end 560 and the second end 570.

The receiving space 750 is configured to at least partially receive the housing 204, comprising in particular the upper part of the rear face 222, up to the edge 550. In an embodiment, the receiving space 750 is complementary in shape to the housing, in particular to the upper part of the rear face 222 of the housing 204, with, for example, a functional clearance when the device 100 is installed in the toilet bowl 106.

In an embodiment, as shown in FIGS. 5 to 8, receiving space 750 has a rounded shape, particularly complementary to the pebble shape of housing 204. Alternatively, as shown in FIG. 9, the receiving space 750 has an angular shape, particularly complementary to the parallelepiped shape of the housing 204.

In an embodiment, the proximal attachment portion 710, and more particularly the second end 570, comprises a stop 610. The stop 610 defines an end of the receiving space 750 opposite the end of the receiving space cooperating with the attachment element 510. The stop 610 is configured to block a movement of the urine analysis device 100 authorized by the fastening arm 600. This blocking is achieved in particular by contact with the front face or transition face.

### Distal Attachment Portion

Fastening arm 600 further comprises a distal attachment portion 720. The distal attachment portion 720 is opposite the proximal attachment portion 710. As seen in FIG. 6, the distal attachment portion 720 is configured to cooperate with the toilet bowl 106.

In an embodiment illustrated in the figures, the distal attachment portion 720 comprises a hook. In particular, the distal attachment portion 720 is movable from a resting configuration, as illustrated in FIGS. 5 and 7, in which the distal attachment <portion 720 is folded upon itself to a deployed configuration, as illustrated in FIG. 6, in which the distal attachment portion 720 is deployed. In the deployed configuration, the distal attachment portion 720 is configured to hook onto the rim 540 of the toilet bowl 106. The distal attachment portion 720 comprises three successive elongated parts 620, 630, 640. As shown in FIG. 7, in the resting configuration, the third part 640 is folded between the first part 620 and the second part 630. The three parts 620, 630, 640 are then substantially parallel to each other. As shown in FIG. 6, in the deployed position, the second part 630 and the third part 640 are spaced apart from the first part 620, each pair of successive parts forming a substantially right angle with each other.

The third part 640 may comprise an anti-slip block 650, for example made of rubber, to improve adhesion of the fastening arm 600 to the bowl 106.

In a variant not shown, the fastening arm 600 comprises a magnet suitable for cooperating with another magnet placed in particular on the attachment element 510.

In a variant not shown, the fastening arm 600 comprises an adhesive part suitable for cooperating with the attachment element 510.

### Intermediate Portion

Fastening arm 600 further comprises an intermediate portion 730 arranged between proximal attachment portion 710 and distal attachment portion 720.

The proximal attachment portion 710 is connected to the intermediate portion 730 at the second end 570. In this configuration, the fastening arm 600 has no branching sections, but only one after the other.

The intermediate portion 730 and the proximal attachment portion 710 form an inflection zone between them. In other words, the fastening arm 600 has a change of direction between the intermediate portion 730 and the proximal attachment portion 710. In other words, the intermediate portion 730 and the proximal attachment portion 710 form an edge between them. In particular, the stop 610 is formed by this change of direction. In particular, with reference to FIG. 7, the intermediate portion 730 and the proximal attachment portion 710 form an angle a of less than 90°, in particular less than 60°, between them at the level of their contact. This improves the mechanical preload of the fastening arm against the rim of the toilet, thereby increasing installation rigidity and compensating for minor tolerances in toilet bowl shapes.

The intermediate portion 730 extends as an extension of the housing 204 in the median plane XZ of the housing 204. In particular, the intermediate portion 730 comprises a rear surface 660 facing the inner wall 112 of the bowl 106 and a front surface 670 opposite the rear surface 660. The front surface 670 extends in line with the front face 220 of the housing, in particular to promote the passage of urine over the housing. In other words, the plane tangent to the housing at stop 610 is substantially parallel to the plane tangent to the front surface 670 of intermediate portion 730 at stop 610.

As can be seen from the figures, the intermediate portion 730 can have a rounded shape. In particular, intermediate portion 730 can have a convex shape, i.e. curved towards the bowl. The convex shape of the intermediate portion 730 tends to move the device 100 away from the bowl 106, as can be seen in FIG. 6.

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

The articles “a” and “an” may be employed in connection with various elements and components, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

1. A urine analysis assembly comprising:
a urine analysis device configured to be placed entirely within a toilet bowl and comprising:
a housing, the housing comprising:
a front face designed to receive a flow of urine from a user urinating in the toilet bowl, and
a rear face opposite the front face;
an attachment element located on the rear face of the housing,
a fastening arm configured to position the urine analysis device entirely within the toilet bowl, the fastening arm comprising a proximal attachment portion configured to cooperate with the attachment element of the urine analysis device,
wherein the proximal attachment portion forms a receiving space for the housing, wherein the receiving space extends from the rear face to beyond an edge of the rear face.

2. The urine analysis assembly according to claim 1, wherein the proximal attachment portion extends between a first end and a second end of the proximal attachment portion, wherein the first end is flush with the attachment element on the rear face and the second end is beyond the edge of the rear face.

3. The urine analysis assembly according to claim 2, wherein the first end extends to the front face of the housing.

4. The urine analysis assembly according to claim 2, wherein the second end comprises a stop configured to block movement of the urine analysis device otherwise permitted by the fastening arm.

5. The urine analysis assembly according to claim 1, wherein the receiving space is of complementary shape to the housing,

6. The urine analysis assembly according to claim 1, in which the receiving space is of complementary shape to the housing with a functional clearance.

7. The urine analysis assembly according to claim 1, wherein the attachment element is arranged in the upper part of the rear face.

8. The urine analysis assembly according to claim 7, in which the attachment element is arranged between 15% and 25% from the edge of the rear face.

9. The urine analysis assembly according to claim 1, wherein the fastening arm further comprises a distal attachment portion configured to cooperate with the toilet bowl.

10. The urine analysis assembly according to claim 7, wherein the fastening arm further comprises an intermediate portion arranged between the proximal attachment portion and the distal attachment portion.

11. The urine analysis assembly according to claim 10, wherein the proximal attachment portion extends between a first end and a second end of the proximal attachment portion, wherein the first end is flush with the attachment element on the rear face and the second end is beyond the edge of the rear face, wherein the proximal attachment portion is connected to the intermediate portion at the second end.

12. The urine analysis assembly according to claim 10, wherein the fastening arm has a change of direction between the intermediate portion and the proximal attachment portion.

13. The urine analysis assembly according to claim 10, wherein the intermediate portion and the proximal attachment portion form an angle between them at a level of their contact of less than 90°.

14. The urine analysis assembly according to claim 10, wherein the intermediate portion and the proximal attachment portion form an angle between them at a level of their contact of less than 60°.

15. The urine analysis assembly according to claim 10, wherein the intermediate portion extends as an extension of the housing in a median plane of the housing.

16. The urine analysis assembly according to claim 10, wherein the intermediate portion has a convex shape.

17. The urine analysis assembly according to claim 1, wherein the rear face comprises at least one spacer projecting from the rear face and configured to contact the toilet bowl.
